(19) United States
(12) Patent Application Publication
(10) Pub. No.: US 2025/0241668 A1
(43) Pub. Date: Jul. 31, 2025

(54) THROMBECTOMY DEVICES INCLUDING THROMBUS DISLODGER ASSEMBLY AND NET ASSEMBLY

(71) Applicant: Bard Peripheral Vascular, Inc., Franklin Lakes, NJ (US)

(72) Inventors: Michaella Kavanagh, Scottsdale, AZ (US); Sam Shreve, Scottsdale, AZ (US)

(73) Assignee: Bard Peripheral Vascular, Inc., Franklin Lakes, NJ (US)

(21) Appl. No.: 18/853,956

(22) PCT Filed: Apr. 22, 2022
(86) PCT No.: PCT/US22/25940

§ 371 Date: Oct. 3, 2024

(51) Int. Cl. A61B 17/221; A61B 17/00; A61B 17/22; A61B 17/32; A61B 17/3207

(52) U.S. Cl. CPC A61B 17/221; A61B 17/00234; A61B 17/320758; A61B 2017/00238; A61B 2017/00305; A61B 2017/00477; A61B 2017/00862; A61B 2017/22072; A61B 2017/2215; A61B 2017/320064

(57) ABSTRACT

A venous thrombectomy device includes a net assembly and a dislodger assembly that are operated independent of each other. The net assembly includes a wire and an expandable net secured at a distal end of the wire, wherein, when in an expanded state, the expandable net defines an interior collection volume with a mouth that opens proximally when the net is advanced distally beyond the thrombus and expanded. The dislodger assembly includes at least one node secured at a distal end of a shaft, wherein the wire of the net assembly extends through a lumen of the shaft such that the dislodger assembly may translate axially over and rotate about the wire, relative to the net.

### TECHNICAL FIELD

The present disclosure relates generally venous thrombectomy, more particularly, to a device and method for dislodging and capturing thrombus.

### BACKGROUND

When left untreated, a blood clot (or thrombus) may “break off” and travel to the lungs (pulmonary embolism) or to the brain (stroke). A blood clot also decreases or blocks blood flow, which may cause damage to surrounding tissues or organs. Blood clots are often treated with medication, such as blood thinners, but in some cases may be too large or pose immediate risk to the patient.

Another treatment is a thrombectomy, which is a surgical procedure used to remove blood clot from inside arteries and veins. This procedure may involve using a catheter to pass a small tube through a tiny incision into the clot site, where special instruments such as a balloon(s) may be used to remove the clot. However, such procedure may require several passes of the balloon to extract all of the thrombus without leaving residual thrombus and, therefore, this procedure is rather time consuming (i.e., lasting as long as an hour) to the detriment of the patient. Moreover, even if accompanied by aspiration, it is possible for parts of the thrombus to flow downstream (distal from the balloon) away from the site (i.e., to the lungs of the brain), which may result in serious medical complications.

### SUMMARY

In one embodiment, a device for removing a thrombus from a vein includes a net assembly including a wire and an expandable net secured at a distal end of the wire, wherein, when in an expanded state, the expandable net defines an interior collection volume with a mouth configured to open proximally when the net is advanced distally, in particular beyond the thrombus, and expanded; and a dislodger assembly having a shaft and at least one node secured at a distal end of the shaft, wherein the wire of the net assembly extends through a lumen of the shaft such that the dislodger assembly is translatable axially over and rotatable about the wire, relative to the net.

In another embodiment, a device for removing a thrombus from a vein includes a net assembly including a wire and an expandable net secured at a distal end of the wire; a dislodger assembly including a shaft and at least one node secured at a distal end of the shaft, wherein the wire of the net assembly extends through a lumen of the shaft such that the dislodger assembly is translatable axially over and rotatable about the wire, relative to the expandable net; and a delivery catheter configured to house the net assembly and the dislodger assembly when the expandable net and the at least one node are in compressed states, wherein the expandable net is independently deployable from the delivery catheter relative to the at least one node such that the device is configured that, when advanced distally beyond the thrombus, the expandable net defines an interior collection volume with a proximally opening mouth.

In yet another embodiment, a device for removing a thrombus from a vein includes a net assembly including a wire and an expandable net secured at a distal end of the wire; a dislodger assembly including a shaft and at least one node secured at a distal end of the shaft, wherein the wire of the net assembly extends through a lumen of the shaft such that the device is configured such that the dislodger assembly is translatable axially over and rotatable about the wire, relative to the net; and a delivery catheter configured to house the net assembly and the dislodger assembly, wherein the net and the at least one node are independently deployable from the delivery catheter, wherein the device is configured such that the net and the at least one node are each in a compressed state when housed in the delivery catheter prior to deployment and the net and the at least one node each self-expand into an expanded state upon deployment, and wherein a distal end of the delivery catheter includes a compliant material that is expandable to receive the thrombus.

These and additional features provided by the embodiments described herein will be more fully understood in view of the following detailed description, in conjunction with the drawings.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a side view of a thrombectomy device according to one or more embodiments of the present disclosure.

FIG. 2 is a side view of a net assembly of the thrombectomy device of FIG. 1.

FIG. 3 is a side view a dislodger assembly of the thrombectomy device of FIG. 1.

FIGS. 4A and 4B are side views of alternate dislodger assemblies utilizable with the thrombectomy device of FIG. 1.

FIGS. 5A-5C are cross-sectional side views of the thrombectomy device of FIG. 1 utilizable with a delivery catheter depicting example deployment.

FIGS. 6A-6C are cross-sectional side views of the thrombectomy device and delivery catheter of FIGS. 5A-5C depicting example operation.

### DETAILED DESCRIPTION

The embodiments described herein provide a thrombectomy device with a net assembly and dislodger assembly that may be deployed and manipulated independent from each other. As used herein, the term “independent” means that the net assembly may be manipulated without manipulation of the dislodger assembly and that dislodger assembly may be manipulated without manipulation of the net assembly, or, stated differently, that the net assembly is separately movable from the dislodger assembly and that the dislodger assembly is separately movable from the net assembly. Other embodiments described herein provide a delivery catheter from which the thrombectomy device may be deployed, with the net assembly and the dislodger assembly being independently deployable and retractable relative thereto. By incorporating a dislodger assembly that is independent of the net assembly capturing residual thrombus, the thrombectomy procedure may be performed more rapidly, as the dislodger assembly may pass multiple times within the vessel without having to remove the device until the end of the procedure.

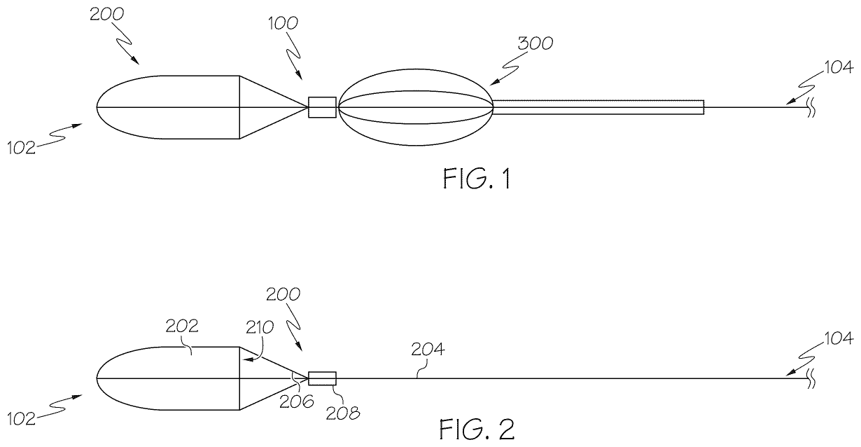

FIG. 1 is side view of a thrombectomy device 100 (hereinafter, the device 100), according to one or more embodiments of the present disclosure. The device has a distal end 102 and a proximal end 104. The device 100 includes a net assembly 200 and a dislodger assembly 300. In the illustrated example, the net assembly 200 is arranged proximate the distal end 102 and the dislodger assembly 300 is arranged proximal to the net assembly 200. As hereinafter described, the net assembly 200 and the dislodger assembly 300 may be moved independently of each other, meaning the dislodger assembly 300 may be moved independent of (or relative to) the net assembly 200 and the net assembly 200 may be moved independent of (or relative to) the dislodger assembly 300.

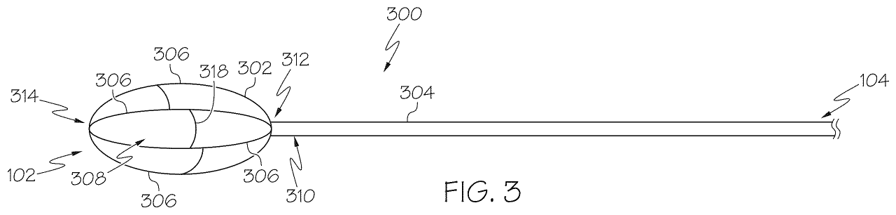

FIG. 2 illustrates the net assembly 200 of FIG. 1 in isolation, according to one or more embodiments of the present disclosure. The net assembly 200 includes a net 202 attached to a wire 204. The wire 204 extends longitudinally, between the distal end 102 and the proximal end 104, with the net 202 being attached thereto at the distal end 102 of the wire 204. The net 202 may include attachment wires 206 that extend proximally from the net 202 and which are connected to the wire 204 via a fastener 208. The fastener 208 may be moveable on the wire 204 to allow flexibility of the net 202 during expansion or retraction. For example, the wire 204 may extend through a center of the net 202 and be fixed to a distal end of the net and, in these examples, the fastener 208 may move along the wire 204 to facilitate expansion and contraction of the net 202. However, in some embodiments, the wire 204 may also stop at the fastener 208 (i.e., the wire 204 may not extend distally beyond the fastener 208). In embodiments where the wire 204 does not run through the center of the net 202 and connect to the distal end of the net 202, then the connection point 208 need not be movably provided on the wire 204. The fastener 208 might also be made with more radiopaque materials (e.g., cobalt chromium, tantalum, iridium platinum, barium sulphate if polymer, etc.) to facilitate easier visualization of the location of the net 202 during a procedure. The net 202 may be made from a self-expanding material. For example, the net 202 could be constructed from a Nitinol mesh, a metallic mesh, or a polymer scaffold.

The net 202 defines an open interior or basket (obscured from view). The basket of the net 202 has a closed distal end and an open mouth 210 at a proximal end. Thus, the open mouth 210 opens in a proximal direction facing the dislodger assembly 300. As described herein, this orientation allows the interior basket of the net 202 to capture and retain thrombus or particles thereof that may flow downstream during a thrombectomy, while also allowing blood to flow there-through without blocking blood flow.

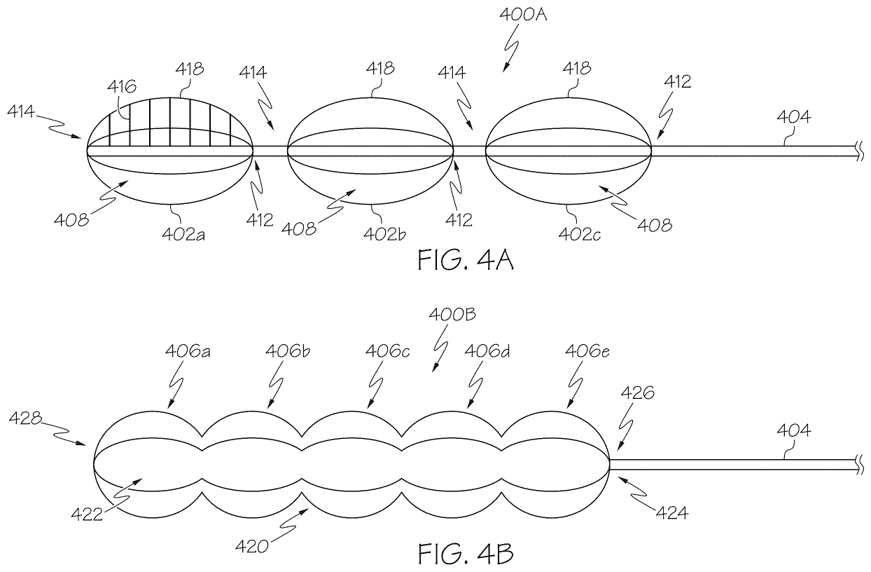

FIG. 3 illustrates the dislodger assembly 300 of FIG. 1, according to one or more embodiments of the present disclosure. The dislodger assembly 300 includes a dislodger or node 302 connected to a guide shaft 304. The shaft 304 extends longitudinally, between the distal end 102 and the proximal end 104, with the node 302 being attached thereto at the distal end 102 of the shaft 304. Here, the shaft 304 is hollow, and defines an interior lumen (not illustrated) through which the wire 204 of the net assembly 200 may extend. In embodiments, the shaft 304 may be constructed of a single polymer such as a polyamide, pebax, polyimide, or some other single material. In other embodiments, the shaft 304 may be constructed of a multilayer material such as a layered polymer, or a polymer inner layer with a stainless steel braid, helix, or wrap outer layer, or a polymer outer layer with a stainless steel braid, helix, or wrap inner layer, etc. In embodiments, the shaft 304 may be constructed of a flexible metal spiral wound tube. Thus, the dislodger assembly 300 may slide distally or proximally over the wire 204 of the net assembly 200 and may also rotate about the wire 204 of the net assembly 200, thereby permitting relative translation and/or rotation between the net assembly 200 and the dislodger assembly 300. By providing the node 302 on a guide shaft 304 that is independently movable relative to the net assembly 200, it is possible to first deploy the net 202 and then move and deploy the node 302 independent of the deployed net 202.

The node 302 is comprised of a plurality of perimeter struts 306, which together define an interior open space 308 of the node 302. In the illustrated example of FIG. 3, a distal end 310 of the shaft 304 is connected to the node 302 at a proximal end 312 of the node 302. As mentioned above, the shaft 304 is hollow and, in this example, the lumen of the shaft 304 communicates with (opens into) the interior open space 308 of the node 302, such that the wire 204 of the net assembly 200 is extendable through the lumen of the shaft 304 and the interior open space 308 of the node 302. However, in other examples, the shaft 304 may extend through the interior open space 308 of the node 302, where (at least) both the proximal end 312 of the node 302 and a distal end 314 of the node 302 are connected to the shaft 304.

The node 302 may have a scaffold like structure. For example, in some embodiments, one or more interior struts 318 may be provided that interconnect neighboring perimeter struts 306. However, the interior struts 318 may extend further about the perimeter of the node 302 so as to interconnect more than two of the perimeter struts 306. The interior struts 318 facilitate breaking apart, cutting, or macerating the thrombus into particles, and to capture and entrain the thrombus or particles thereof within the interior open space 308 of the node 302. As described below, one or more additional struts may be provided to interconnect the perimeter struts 306 to the shaft 304.

The perimeter struts 306, and the one or more interior struts 318 (if any), also define an exterior shape or appearance of the node 302. FIGS. 1 and 3 illustrate the node 302 having an oval shape. However, it will be appreciated that the node 302 may have various other geometries, such as circular shapes, conical shapes, cylindrical shapes, conical-cylindrical shapes (i.e., a cylinder with a cone at each circular end face), etc. Moreover, FIGS. 1 and 3 illustrate the dislodger assembly 300 as having a single node. However, it will be appreciated that the dislodger assembly 300 may have more than one node. For example, it may include two (2) or more nodes. Furthermore, any one or more of the nodes 302 may be connected together, so as to have two (2) or more bulbous portions.

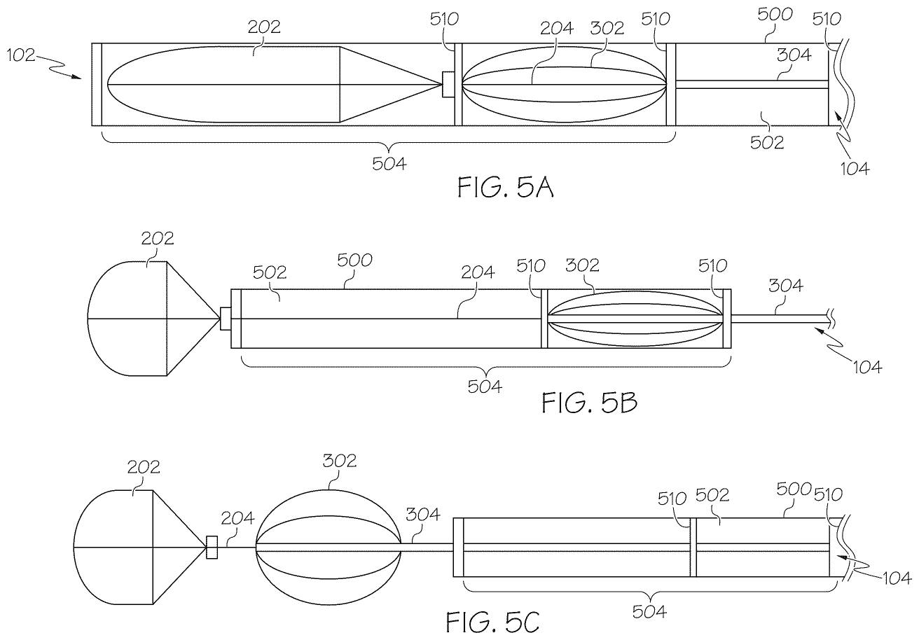

FIGS. 4A and 4B illustrate alternate embodiments of the dislodger assembly, according to one or more alternate embodiments of the present disclosure. In particular, FIG. 4A illustrates an example dislodger assembly 400A that includes a plurality of disconnected nodes 402a,402b,402c, with the disconnected node 402c being the distal-most node of the plurality of disconnected nodes 402a,402b,402c. However, more or less than three (3) of the disconnected nodes 402a,402b,402c may be provided such that the disconnected node 402c may not be the distal-most node. Also in this example, a shaft 404 extends through an interior open space 408 each of the disconnected nodes 402a,402b,402c, such that each of the disconnected nodes 402a,402b,402c is connected to the shaft 404 at respective proximal and distal end 412, 414 of each of the disconnected nodes 402a,402b,402c, as described above. FIG. 4A also illustrates an example one or more interior support struts 416 may be provided that interconnect any one or more of perimeter struts 418 to the shaft 404. The interior support struts 416 may facilitate breaking apart, cutting, or macerating the thrombus into particles, and to capture and entrain the thrombus or particles thereof within interior open space 408 of the node 402. While FIG. 4A illustrates just the first disconnected node 402a as having the interior support struts 416, any one or more of the disconnected nodes 402a,402b,402c may also include the interior support struts 416. Also, any one or more of the disconnected nodes 402a,402b,402c may also include the interior struts 318 (FIG. 3) in addition to or in lieu of the interior support struts 416.

FIG. 4B illustrates an example dislodger assembly 400B comprising a node 420 that is configured with a plurality of bulbous portions 406a,406b,406c,406d,406e, according to one or more embodiments. In the illustrated example, the bulbous portions 406a,406b,406c,406d,406e are connected to each other so as to define an individual node element having an individual interior open space 422, but having the functionality of multiple discrete nodes. Also, more or less than five (5) of the connected bulbous portions may be provided. In the illustrated example, a distal end 424 of the shaft 404 is connected to a proximal end 426 of the node 420 (i.e., to a proximal end of the bulbous portion 406e of the node 420), such that the node 420 is supported at the distal end 424 of the shaft 404. Here, the lumen within the shaft 404 communicates with (opens into) the interior open space 422 of the node 420. However, in other examples, the shaft 404 may extend through the interior open space 422 of the node 420, with (at least) both the proximal end 426 of the node 420 and a distal end 428 of the node 420 being connected to the shaft 404. Also, in some examples, the shaft 404 may extend partially through the interior open space 422 of the node 420, such that it only extends within the interior open space 422 corresponding with less than all of the bulbous portions 406a,406b,406c,406d,406e.

Regardless of its structural configuration, any one or more of the nodes 302, 402, 420 may be made from a self-expanding material, such as a Nitinol mesh, a metallic mesh, or a polymer scaffold.

During a thrombectomy procedure, a catheter may be utilized to deliver and retract the device 100 to and from the surgical site.

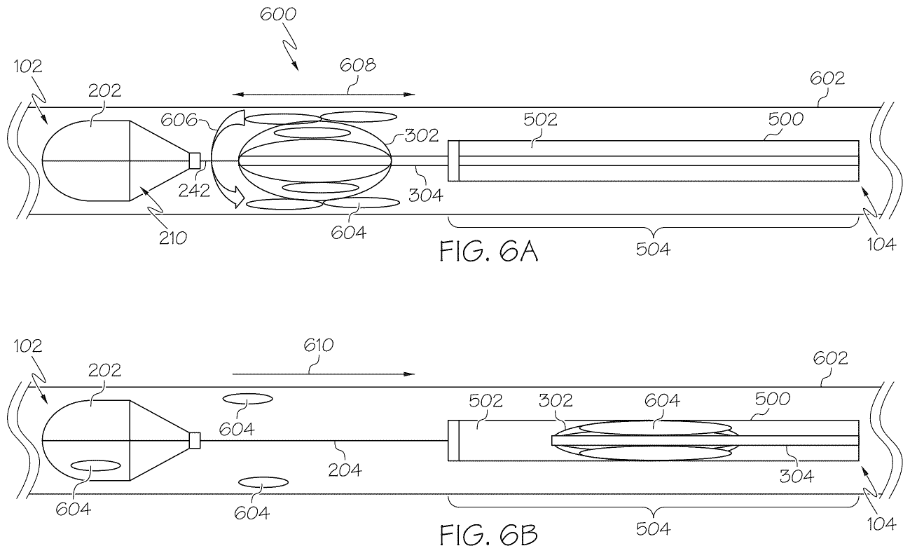

FIGS. 5A-5C are cross-sectional side views of the thrombectomy device of FIG. 1 utilizable with an exemplary delivery catheter 500, according to one or more embodiments of the present disclosure. As hereinafter described, the delivery catheter 500 is utilizable for delivering the device 100 to the intervention site, deploying and retracting the net assembly 200 and the dislodger assembly 300 at the intervention site, and then retracting the device 100 back away from the intervention site and out of the patient's body.

In particular, FIG. 5A illustrates the net assembly 200 and the dislodger assembly 300 stowed in a lumen 502 of the delivery catheter 500. In the illustrated example, the delivery catheter 500 includes a distal portion 504 configured to retain and house the net 202 and the node(s) 302. As hereinafter described, the distal end 504 of the delivery catheter 500 may be made of a compliant material that can deform and expand to allow storage of the thrombosis. Accordingly, the distal end 504 of the delivery catheter 500 may expand when the dislodger assembly 300 carrying the thrombus is distally pulled back within the distal end of the delivery catheter 500, such that there is adequate space to receive the thrombus entrained in the node(s) 302.

In embodiments, the delivery catheter 500 may include one or more internal valves 510 to help trap the thrombus in the catheter 500 when the node 302 is withdrawn. For example, when the node 302 pulls the thrombus into the sheath defined by the catheter 500, the one or more internal valves 510 will help maintain the thrombus in place if the node 302 was redeployed to capture more thrombus. These were included in an earlier version of the idea that we decided to remove.

FIG. 5B illustrates how the net assembly 200 may be deployed independently of the dislodger assembly 300. Here, the wire 204 may be advanced distally, through the shaft 304 and the node(s) 302 of the dislodger assembly 300, to thereby advance or extend the net 202 connected thereto out of the distal end 504 of the delivery catheter 500. During this extension of the net assembly 200, the dislodger assembly 300 may remain stationary within the catheter 500. FIG. 5A illustrates the net 202 is in a compressed or unexpanded state when located within the distal end 504 of the delivery catheter 500; however, as illustrated in FIG. 5B, upon exiting the distal end 504 of the delivery catheter 500, the net 202 may expand into an expanded state such that the net 202 is operable to filter the flowing blood and collect or capture thrombus particles.

FIG. 5C illustrates how the dislodger assembly 300 may be deployed independently of, and after, the previously deployed net assembly 200. Here, the net 202 has already been deployed and expanded into the expanded state, distal of the dislodger assembly 300, such that the shaft 304 and the node(s) 302 may be advanced in a distal direction, over the wire 204 of the net assembly 200. As described below, the dislodger assembly 300 may continue to be advanced distally along the wire 204 until the node(s) 302 contacts or passes at least partially through the thrombus. Then, the dislodger assembly 300 may be manipulated so as to break apart and entrain the thrombus within the interior open space 308 of the node(s) 302. For example, the shaft 304 may be repeatedly distally pushed and proximally pulled, such that the node(s) 302 translate distally and proximally through the thrombus, thereby breaking apart and/or capturing the thrombus within the node(s) 302. Also, the shaft 304 may be rotated about the wire 204, such that the node(s) 302 correspondingly rotates, thereby braking apart and/or capturing the thrombus within the node(s) 302. FIGS. 5A and 5B illustrate the node(s) 302 in a compressed or unexpanded state when located within the distal end 504 of the delivery catheter 500; however, as illustrated in FIG. 5C, upon exiting the distal end 504 of the delivery catheter 500, the node(s) 302 may expand into an expanded state such that the node(s) 302 is operable to cleave the thrombus and collect or capture thrombus particles within the interior open space 308 of the node(s) 302.

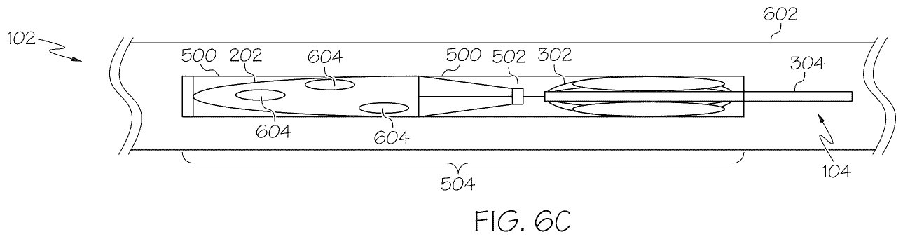

FIGS. 6A-6C illustrate an example thrombectomy procedure utilizing the device 100 and the delivery catheter 500 of FIGS. 5A-5C. In particular, FIG. 6A depicts an obstruction or blockage 600 within an artery or vein 602, wherein the blockage comprises a thrombus or blood clot 604. Here, the net assembly 200 has been deployed such that the net 202 has been distally advanced to a location that is distal relative to the blockage 600, such that the open mouth 210 opens towards (or faces) the blockage 600 such that any distally flowing pieces of the thrombus 604 will be caught in the net 202. To reach this location, the net 202 was distally advanced through the blockage 600.

Also, FIG. 6A illustrates the dislodger assembly 300 having been deployed such that the node 302 is located or positioned at the blockage 600. In particular, the shaft 304 has been distally translated to thereby distally push the node 302 into contact with at least a portion of the thrombus 604. Here, the node 302 has been positioned at a location that substantially corresponds with the location of the blockage 600, such that the node 302 is substantially co-extensive with the thrombus 604, as evaluated along the length of the artery or vein 602. Thereafter, the dislodger assembly 300 may be rotated about an axis defined by the shaft 304, as indicated by rotation arrow 606, to thereby agitate the thrombus 604 and break it apart into one or more pieces or particles of the thrombus 604. Also, by rotating the node 302 about the rotation arrow 606, the thrombus 604 (or particles of it) may become capture or entrained within the interior open space 308 of the node 302. Furthermore, the dislodger assembly 300 may be translated along the axis defined by the shaft 304, as indicated by translation arrow 608, to thereby agitate the thrombus 604 and break it apart into one or more pieces or particles of the thrombus 604, and to further capture or entrained it within the interior open space 308 of the node 302. In addition, such translation of the dislodger assembly 300 may be beneficial for repositioning the node 302 as may be beneficial depending on the size (length) of the blockage 600 to be treated. Also, while the dislodger assembly 300 has been described as being rotated first and then translated second to macerate the thrombus 604, it may occur in reverse order. Moreover, there may be more than rotations and/or more than one translations of the node 302 occurring in various orders during a single operation. Regardless, the dislodger assembly 300 may be manipulated such that the node 302 moves as indicated by the arrows 606,608, which breaks apart the thrombus 604 and entrains at least a portion of the thrombus 604 within the interior open space 308 of the node 302.

Thereafter, the dislodger assembly 300 entraining at least some of the thrombus 604 may be retracted back within the catheter 500, as depicted in FIG. 6B. Here, the dislodger assembly 300 has been translated in a proximal direction 610 such that the node 302, which has entrained or captured some pieces of the thrombus 604, is located within the distal end 504 of the delivery catheter 500. Because the node 302 is expandable, it is able to compress (even when entraining the thrombus 604) into a compressed state as it enters the catheter 500, to thereby facilitate removal from the artery or vein 602. Also, as mentioned above, the distal portion 504 of the delivery catheter 500 may be made of a compliant material such that it can expand to facilitate storage within the delivery catheter 500 of the node 302 with the thrombosis 604 entrained therein. As shown, a few pieces of the thrombus 604 (i.e., residual thrombus) were not capture and entrained by the node 302 and remain in the artery or vein 602. Also, at least one of these residual pieces of the thrombus 604 flowed downstream from the site of the blockage 600 and into the open mouth 210 of the expanded net 202, such that it was capture by the net 202 and prevented from causing complications (i.e., embolism).

Thereafter, the net assembly 200 may be retracted back within the catheter 500, as depicted in FIG. 6C. As the net 202 is retraced in the proximal direction 610, it carries with it the piece of the thrombus 604 that it captured in FIG. 6B. However, as the net 202 continues to be retracted in the proximal direction 610, it may capture any other remaining pieces of the thrombus 604 that had not previously been captured by the node 302 and/or the net 202 as described with reference to FIGS. 6A and 6B. Because the net 202 is also expandable, it is able to compress (even when carrying the thrombus 604) into a compressed state as it enters the catheter 500, to thereby facilitate removal from the artery or vein 602. Also, as mentioned above, the distal portion 504 of the delivery catheter 500 may be made of a compliant material such that it can expand to facilitate storage within the delivery catheter 500 of the net 202 with the thrombosis 604 therein.

Utilizing the disclosed thrombectomy devices, which incorporate a dislodger assembly and a net assembly that are independently operable relative to each other, the physician to perform thrombectomy procedures more rapidly and efficiently with less risk to the patient. The disclosed thrombectomy devices permit the physician to deploy the net assembly distal of the thrombus and then perform multiple passes with the dislodger assembly within the vessel, with the net assembly being deployed to catch any thrombus portions that may flow downstream, without having to remove the device until the end of the procedure.

Embodiments can be described with reference to the following numbered clauses, with certain features laid out in the dependent clauses.

Clause 1: A device for removing a thrombus from a vein, comprising: a net assembly comprising a wire and an expandable net secured at a distal end of the wire, wherein, when in an expanded state, the expandable net defines an interior collection volume with a mouth configured to open proximally when the net is advanced distally, in particular beyond the thrombus, and expanded; and a dislodger assembly having a shaft and at least one node secured at a distal end of the shaft, wherein the wire of the net assembly extends through a lumen of the shaft such that the dislodger assembly is translatable axially over and rotatable about the wire, relative to the net.

Clause 2: The device of clause 1 further comprising a delivery catheter configured to house the net assembly and the dislodger assembly, optionally wherein the net and the at least one node are independently deployable from the delivery catheter.

Clause 3: The device of clause 2, wherein the device is configured such that the net and the at least one node are each in a compressed state when housed in the delivery catheter prior to deployment.

Clause 4: The device of any of clauses 1-3, wherein the device is configured such that the net and/or the at least one node each self-expand into an expanded state upon deployment.

Clause 5: The device of any of clauses 1-4, wherein the net is self-expanding.

Clause 6: The device of any of clauses 1-5, wherein the at least one node is self-expanding.

Clause 7: The device of any of clauses 1-6, wherein the at least one node comprises a plurality of unconnected nodes.

Clause 8: The device of any of clauses 1-7, wherein the at least one node comprises a plurality of connected nodes.

Clause 9: The device of any of clauses 1-8, wherein the at least one node comprises a plurality of perimeter struts that together define an interior space of the at least one node for containing and entraining thrombus.

Clause 10: The device of clause 9, wherein the at least one node comprises a plurality of interior struts that each interconnect two or more of the perimeter struts.

Clause 11: The device of any of clauses 9-10, wherein the at least one node comprises a plurality of interior supports that each interconnect the shaft to one or more of the perimeter struts.

Clause 12: The device of any of clauses 1-8, wherein the shaft extends through each of the at least one node, and the at least one node comprises a plurality of perimeter struts extending from the shaft that together define an interior space of the at least one node for containing and entraining thrombus.

Clause 13: The device of clause 12, wherein the at least one node comprises a plurality of interior struts that each interconnect two or more of the perimeter struts and/or the at least one node comprises a plurality of interior supports that each interconnect the shaft to one or more of the perimeter struts.

Clause 14: A device for removing a thrombus from a vein, comprising: a net assembly comprising a wire and an expandable net secured at a distal end of the wire; a dislodger assembly comprising a shaft and at least one node secured at a distal end of the shaft, wherein the wire of the net assembly extends through a lumen of the shaft such that the dislodger assembly is translatable axially over and rotatable about the wire, relative to the expandable net; and a delivery catheter configured to house the net assembly and the dislodger assembly when the expandable net and the at least one node are in compressed states, wherein the expandable net is independently deployable from the delivery catheter relative to the at least one node such that the device is configured that, when advanced distally beyond the thrombus, the expandable net defines an interior collection volume with a proximally opening mouth.

Clause 15: The device of clause 14, wherein the at least one node is in a compressed state when housed in the delivery catheter prior to deployment.

Clause 16: The device of any of clauses 14-15, wherein the expandable net and the at least one node each self-expand into an expanded state upon deployment.

Clause 17: The device of any of clauses 14-16, wherein a distal end of the delivery catheter comprises a compliant material that is expandable to receive the thrombus.

Clause 18: The device of clause 17, wherein the compliant material is silicone.

Clause 19: A device for removing a thrombus from a vein, comprising: a net assembly comprising a wire and an expandable net secured at a distal end of the wire; a dislodger assembly comprising a shaft and at least one node secured at a distal end of the shaft, wherein the wire of the net assembly extends through a lumen of the shaft such that the device is configured such that the dislodger assembly is translatable axially over and rotatable about the wire, relative to the net; and a delivery catheter configured to house the net assembly and the dislodger assembly, wherein the net and the at least one node are independently deployable from the delivery catheter, wherein the device is configured such that the net and the at least one node are each in a compressed state when housed in the delivery catheter prior to deployment and the net and the at least one node each self-expand into an expanded state upon deployment, and wherein a distal end of the delivery catheter comprises a compliant material that is expandable to receive the thrombus.

Clause 20: The device of clause 19, wherein, when in an expanded state, the expandable net defines an interior collection volume with a mouth configured to open proximally when the net is advanced distally beyond the thrombus and expanded.

The device of Clauses 19 and/or 20 may be configured according to Clauses 1 to 13 and/or Clauses 14 to 18.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

The terms “proximal” and “distal” are defined herein relative to a surgeon utilizing the venous thrombectomy device. The term “proximal” refers to the position of an element closer to the surgeon and the term “distal” refers to the position of an element further away from the surgeon. Moreover, the use of directional terms such as above, below, upper, lower, upward, downward, left, right, and the like are used in relation to the illustrative embodiments as they are depicted in the figures, the upward or upper direction being toward the top of the corresponding figure and the downward or lower direction being toward the bottom of the corresponding figure.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

1. A device for removing a thrombus from a vein, comprising:
a net assembly comprising a wire and an expandable net secured at a distal end of the wire, wherein, when in an expanded state, the expandable net defines an interior collection volume with a mouth that opens proximally when the net is advanced distally beyond the thrombus and expanded; and
a dislodger assembly comprising a shaft and at least one node secured at a distal end of the shaft, wherein the wire of the net assembly extends through a lumen of the shaft such that the dislodger assembly may translate axially over and rotate about the wire, relative to the net.

2. The device of claim 1, further comprising a delivery catheter configured to house the net assembly and the dislodger assembly, wherein the net and the at least one node are independently deployable from the delivery catheter.

3. The device of claim 2, wherein the net and the at least one node are each in a compressed state when housed in the delivery catheter prior to deployment.

4. The device of claim 3, wherein the net and the at least one node each self-expand into an expanded state upon deployment.

5. The device of claim 1, wherein the net is self-expanding.

6. The device of claim 1, wherein the at least one node is self-expanding.

7. The device of claim 1, wherein the at least one node comprises a plurality of unconnected nodes.

8. The device of claim 1, wherein the at least one node comprises a plurality of connected nodes

9. The device of claim 1, wherein the at least one node comprises a plurality of perimeter struts that together define an interior space of the at least one node for containing and entraining thrombus.

10. The device of claim 9, wherein the at least one node comprises a plurality of interior struts that each interconnect two or more of the perimeter struts.

11. The device of claim 9, wherein the at least one node comprises a plurality of interior supports that each interconnect the shaft to one or more of the perimeter struts.

12. The device of claim 1, wherein the shaft extends through each of the at least one node, and the at least one node comprises a plurality of perimeter struts extending from the shaft that together define an interior space of the at least one node for containing and entraining thrombus.

13. The device of claim 12, wherein the at least one node comprises a plurality of interior struts that each interconnect two or more of the perimeter struts and/or the at least one node comprises a plurality of interior supports that each interconnect the shaft to one or more of the perimeter struts.

14. A device for removing a thrombus from a vein, comprising:
a net assembly comprising a wire and an expandable net secured at a distal end of the wire;
a dislodger assembly comprising a shaft and at least one node secured at a distal end of the shaft, wherein the wire of the net assembly extends through a lumen of the shaft such that the dislodger assembly may translate axially over and rotate about the wire, relative to the expandable net; and
a delivery catheter configured to house the net assembly and the dislodger assembly when the expandable net and the at least one node are in compressed states, wherein the expandable net is independently deployable from the delivery catheter relative to the at least one node such that, when advanced distally beyond the thrombus, the expandable net defines an interior collection volume with a proximally opening mouth.

15. The device of claim 14, wherein the at least one node is in a compressed state when housed in the delivery catheter prior to deployment.

16. The device of claim 15, wherein the expandable net and the at least one node each self-expand into an expanded state upon deployment.

17. The device of claim 14, wherein a distal end of the delivery catheter comprises a compliant material that is expandable to receive the thrombus.

18. The device of claim 15, wherein the compliant material is silicone.

19. A device for removing a thrombus from a vein, comprising:
a net assembly comprising a wire and an expandable net secured at a distal end of the wire;
a dislodger assembly comprising a shaft and at least one node secured at a distal end of the shaft, wherein the wire of the net assembly extends through a lumen of the shaft such that the dislodger assembly may translate axially over and rotate about the wire, relative to the net; and
a delivery catheter configured to house the net assembly and the dislodger assembly, wherein the net and the at least one node are independently deployable from the delivery catheter, wherein the net and the at least one node are each in a compressed state when housed in the delivery catheter prior to deployment and the net and the at least one node each self-expand into an expanded state upon deployment, and wherein a distal end of the delivery catheter comprises a compliant material that is expandable to receive the thrombus.

20. The device of claim 19, wherein, when in an expanded state, the expandable net defines an interior collection volume with a mouth that opens proximally when the net is advanced distally beyond the thrombus and expanded.
